(19) United States
(12) Patent Application Publication
(10) Pub. No.: US 2007/0129426 A1
(43) Pub. Date: Jun. 7, 2007

(54) Therapeutic drugs for arthritis

(75) Inventor: Qiang YU, Shanghai (CN)

Correspondence Address: HAMRE, SCHUMANN, MUELLER & LARSON, P.C., P.O. BOX 2902, MINNEAPOLIS, MN, 55402-0902, US

(73) Assignee: Shanghai Ambrosia Pharmaceutical Co., Ltd., Shanghai (CN)

(21) Appl. No.: 10/584,877

(22) PCT Filed: Dec. 27, 2004
(86) PCT No.: PCT/CN04/01523

§ 371 Date: Jun. 29, 2006

(30) Foreign Application Priority Data
Dec. 30, 2003 (CN) 200310124512.0

(51) Int. Cl. A61K 31/366

(52) U.S. Cl. 514/453

(57) ABSTRACT

The present invention relates to the coumestans compounds extracted from Compositae plants, drug compositions or health products comprising the compounds and the use in treatment of rheumatic arthritis or rheumatoid arthritis, osteoarthritis. The animal experiments in vivo or in vitro indicate that the coumestans compounds can effectively treat and alleviate symptoms of rheumatic arthritis or rheumatoid arthritis, and osteoarthritis.

### TECHNICAL FIELD

The invention relates to the coumestans compounds extracted from Compositae plants and drug compositions comprising the compounds and the use as treatment for rheumatic arthritis or rheumatoid arthritis and osteoarthritis.

### BACKGROUND ART

Arthritis is a chronic inflammation of joints. The cause and the form of the disease are complex. Rheumatoid arthritis (RA) and osteoarthritis are the two major forms of arthritis. RA is a common disease of chronic inflammation of multiple joints. It is characterized by chronic, progressive, symmetric, and invasive deformation of the peripheral joints. Its pathological diagnosis is hyperplasia of synoviocytes, hyperaemia, thickening of blood vessel wall, infiltration of inflammatory cells, hyperplasia, transparency, and degeneration of fibrotic tissues. It is often accompanied by systemic inflammation of other organs. The etiology of rheumatoid arthritis is unknown. Viruses, bacteria, sexual hormone, genetics and environmental factors may associate with rheumatoid arthritis. Other factors such as health conditions may also play a role (William PA. The pathophysiology and treatment of rheumatoid arthritis. Arthritis Rheumatism 1997; 40(4); 585-597), that is, rheumatoid arthritis associates with genetic genes. The following hypotheses have been proposed for the mechanism of RA. (I). HLA may act as a receptor for certain pathogens; (II). Antigen fragments of pathogens may bind amino acids of the HLA molecule; (III). Molecular mimics. Part of HLA may contain sequence homology with certain pathogens. For example, HLA-DR₄ shares sequence homology to EB virus capsid antigen (gp 110), which turns the antibodies for EB pathogen against its own antigen. After entering a human body, the antigens are engulfed by macrophages or macrophage-like cells, then digested, concentrated, and eventually the antigens form a complex with HLA-DR on the cell membrane. If this antigen-HLA complex is recognized by T cell receptor, then T help cells are activated. The activation of T cells will trigger a series immunological response, which includes activation and differentiation of B lymphocytes and mass production of immunoglobulins. One of the immunoglobulins is the rheumatoid factor (RF). 70% of RA patients have RF in their sera. RF is an antibody against the immunoglobulin IgG Fc and binds IgG of its own. The formation of the immunocomplex of RF and IgG is one of the major causes of RA, thus, rheumatoid arthritis is an autoimmune disease. Other alterations, in immunological regulation and in the interactions between subclass lymphocytes, also contribute to the occurrence of the disease. The pathology of such alterations is synovitis, which causes deformation of joints when cartilage and bone are affected.

Rheumatoid arthritis is one of the top five common human diseases. WHO estimated that RA affects 1% of the human population on earth. There are more than 10 million people affected in China. 50% of the RA patients will be disabled in 10 years after onset of the disease and their quality of life will be severely reduced. In severe cases, it can cause deaths (Li K J et al. The rheumatoid arthritis drugs developed in recent years. The international medicine antibiotics chapter. 2003; Jan. 24(1); 24-29).

Osteoarthritis, also known as hypertrophic arthritis, degenerative arthritis, proliferative arthritis or osteoarthroses, is the most common type of arthritis, especially among elders; 50% of the people over age 50 and 80% of the people over age 55. People that are over 65 generally suffer this disease. The cause of osteoarthritis is not entirely clear, however, it may be a combined result of mechanical impairment and physiological alterations with aging, as well as other factors such as, obesity, bone density, injury and genetics.

Most of the arthritis is chronic, progressive, and requires long-term medication. The basic goal of the treatments is to reduce pain and inflammation; to prevent deformation of bone, cartilage, and soft tissues; and to maintain the normal function of the joints; thereby maintaining the normal daily activities of the patients. The conventional treatment for arthritis is a three-step “pyramid” form of medication, including sequentially using first-line, second-line and third-line anti-inflammatory drugs.

The First-Line Drug: Nonsteroidal Anti-Inflammatories (NSAIDs)

Currently, the typical NSAIDs are the major anti-arthritis drugs. They reduce inflammation and pain in the early stage of the disease by inhibiting cyclooxygenase (COX), which catalyses the production of prostaglandin (PG). The NSAIDs are effective in treating the symptoms of the acute arthritis, but have little effect on preventing the progression of the disease. Currently there are many kinds of drug as this, mainly including acemetacin, diclofenac, ibuprofen, indomethacin, meloxicam, ketoprofen, sulindac, auranofin, naproxen, nabumetone, piroxicam, mecolfenamic acid, chlofenamic acid, mefenamic acid, pirprofen, fenbufen, tolmetin, flufenamide acid, fenoprofen, methocarbamol, nimesulide, celecoxib, and rofecoxib. NSAIDs are the first-line anti-arthritis drugs and are critical in treating arthritis. Their effectiveness in treating arthritis remains controversial among the doctors and patients. Their severe adverse effects, however, cannot be overlooked because the drugs listed above inhibit the production of COX1 and COX-2 simultaneously and the usage often brings many severe adverse effects. The most common side effects of NSAIDs include gastrointestinal complex syndrome, upset stomach, abdominal pain, ulcers, gastrointestinal bleeding, as well as damages to kidney, liver, and blood system. The elders are especially prone to the side effects because of the aging in the kidney and gastrointestinal organs. In England, the annual cost for treating rheumatoid arthritis is estimated at about 35 million pounds, whereas the cost for treating the side effects is about 58 million pounds, which is about 3 times of the cost of NSAIDs when the diagnosis and monitory costs are included.

The newly marketed NSAIDs, such as meloxican and aceclofenac, have improved significantly by reducing gastrointestinal adverse effects and their tolerance by patients, though their efficacies are hardly any better.

The Second-Line Drug: Disease-Modifying Anti-Rheumatic Drugs (DMARDS)

These classes of drugs were called “disease modifying” or “second-line drugs” in the past. They are slow-acting drugs on rheumatoid arthritis. It usually takes a certain period of time to become effective, but the effect will last even after discontinuing the medication. The commonly used drugs under this class include methotrexate (MTX), gold preparation, salazosulfadimidine, penicillamine, chloroquine, and tripterygium wilfordii. Certain cytotoxic drugs, such as ciclosporin, cyclophosphamide, methotrexate, and Tripterygium wilfordii, also belong to this class. This class of drugs inhibits inflammation through different routes. They are frequently used as second-line drugs to treat systemic lupus erythematosus, rheumatoid arthritis, and vasculitis. This class of drugs, although has many serious side effects, is particularly effective to improve prognosis of these diseases. One example is leflunomide, which was approved by the FDA in 1998 to be first marketed in the United States as an anti-rheumatoid arthritis drug. Leflunomide is a reversible inhibitor of dihydroorate dehydrogenase, which catalyzes pyrimidine synthesis. It selectively acts on lymphocytes to inhibit their proliferation and therefore slowing down the progression at different stages of the disease. The efficacy of leflunomide is similar or better than that of methotrexate and salazosulfadimidine, and its safety profile and speed of action are improved. Leflunomide, however, can cause diarrhea, alopecia, and rash. The liver function must be monitored due to the increased level of liver enzymes (ALT and AST).

The Third-Line Drug: Corticosteroids.

The corticosteroids are strong anti-inflammation and anti-allergy drugs. They are effective in improving prognosis of pathological changes of connective tissues, but they cannot completely cure these diseases. Their side effects increase as the dose and the length of the treatment increase. It is therefore important to weigh its efficacy carefully against its side effects. The corticosteroids are mainly glucocorticoids such as cortisone and prednisolone.

In addition to the described drugs above, there also exist in biological drugs such as anti-TNF-a antibody, interferon-γ, and RA vaccines. Other methods include Chinese traditional medicine, radiation, and gene therapy (Wang B et al. The progress of anti-rheumatoid arthritis therapy. J. Shanxi Medicine; 2001 Feb. 30 (1) 34-38). Although there are many anti-arthritis drugs on the markets, there is no very effective drug to cure or to control the progression of arthritis, especially for rheumatic/rheumatoid arthritis and osteoarthritis. Many of the NSAIDs have serious gastrointestinal side effects. Their efficiency, particularly the phenylamines, such as acetaminophen, is often not clear. The DMARDs also have serious toxic effects on livers and kidneys. The glucocorticoids, although effective in reducing inflammation, can cause infection, osteoporosis, and dysfunction of adrenal cortex. Therefore, it is recommended that glucocorticoids is not used as a long-term medication. Other anti-arthritis drugs also have unclear efficiency problems, including slow action and severe side effects.

In summary, there is a need for fast-acting and effective pain relief drugs with fewer side effects for arthritis, especially novel substances that has therapeutic value treatment for arthritis, which are derived from natural plants.

### CONTENTS OF THE INVENTION

The objective of the invention is to provide a class of coumestans compounds for treating arthritis effectively.

In the first aspect, the invention provides a use of the coumestans compound of formula I or pharmaceutically acceptable salts thereof, or an extract containing the coumestans compound of formula I or pharmaceutically acceptable salts thereof in the manufacture of a medicament for the treatment or prevention of arthritis

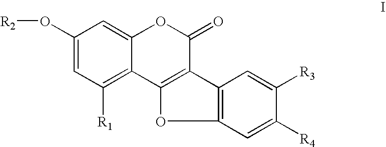

wherein

R₁ represents H, OH, or methoxyl;

R₂ represents H, OH, or C₁-C₈ alkyl;

R₃ and R₄ are each independently selected from the group consisting of H, halogen, OH, and methoxyl.

In a preferred embodiment, the pharmaceutically acceptable salt of the compound of formula I are formed with the acids selected from the group consisting of chlorhydric acid, hydrobromic acid, sulfuric acid, citric acid, tartaric acid, phosphoric acid, lactic acid, pyruvic acid, acetic acid, succinic acid, oxalic acid, fumaric acid, maleic acid, ketosuccinic acid, methane-sulfonic acid, ethyl-sulfonic acid, benzene sulfonic acid, and isethionic acid.

In another preferred embodiment, the compound of formula I or the extract is extracted from the plant Compositae.

In another preferred embodiment, the plant Compositae is selected from Eclipta prostrata Linn, Wedelia chinensis, and Eclipta alba.

In another preferred embodiment, said compound of formula I is wedelolactone as shown in formula II:

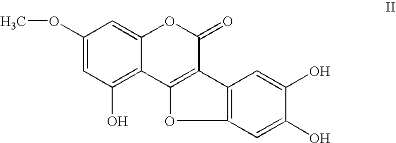

In another preferred embodiment, the arthritis is selected from the group consisting of rheumatic arthritis, rheumatoid arthritis, and osteoarthritis.

In another preferred embodiment, the compound of formula I is produced by total or semi-chemical synthesis, or by biological conversion.

In another preferred embodiment, the compound of formula I is produced by a extraction method comprising the steps of:

(a) extracting the fruits, leaves, or branches of the Compositae plant with 95±3% ethanol, thereby producing ethanol extract;

(b) dissolving the above ethanol extract in 5-300 volumes (preferably 10-200 volumes, and more preferably 20-100 volumes) of H₂O at 50-80° C., filtering to remove the precipitates and collecting the H₂O phase;

(c) extracting the H₂O phase in step (b) with acetic ester and collecting the acetic ester phase;

(d) concentrating and drying the acetic ester phase in step (c) to produce the precipitates;

(e) eluting the precipitates in (d) on a silica gel column with a 5:1 to 1:2 gradient of petroleum ether/acetone mixture and collecting the fraction eluted with 1:1 petroleum ether/acetone;

(f) concentrating the fraction eluted in step (e) to produce concentrated residue;

(g) eluting the concentrated residue in step (e) on a silica gel column with a 5:1 to 1:2 gradient of dichloromethane/acetone mixture and collecting the fraction eluted with 3:1 dichloromethane/acetone;

(h) eluting the fraction eluted in step (g) on a silica gel column with a 20:10:1 to 5:10:1 gradient of toluene-acetone-formate mixture and collecting the fraction eluted with 10:10:1 toluene-acetone-formate;

(i) eluting the fraction eluted in step (h) on a silica gel column with a 30:1 to 1:1 gradient of dichloromethane/methanol mixture and collecting the fraction eluted with 20:1 dichloromethane/methanol; and

(j) recrystallizing the fraction eluted in step (i) with ethanol, thereby producing the coumestans compounds of formula I as precipitates.

In the second aspect, the invention provides a method for producing coumestans, comprising the steps of:

(a) extracting the fruits, leaves, or branches of the Compositae plant with 95±3% ethanol, thereby producing ethanol extract;

(b) dissolving the above ethanol extract in 5-300 volumes of H₂O at 50-80° C., filtering to remove the precipitates and collecting the H₂O phase;

(c) extracting the H₂O phase in step (b) with acetic ester and collecting the acetic ester phase;

(d) concentrating and drying the acetic ester phase in step (c) to produce the precipitates;

(e) eluting the precipitates in (d) on a silica gel column with a 5:1 to 1:2 gradient of petroleum ether/acetone mixture and collecting the fraction eluted with 1:1 petroleum ether/acetone;

(f) concentrating the fraction eluted in step (e) to produce concentrated residue;

(g) eluting the concentrated residue in step (f) on a silica gel column with a 5:1 to 1:2 gradient of dichloromethane/acetone mixture and collecting the fraction eluted with 3:1 dichloromethane/acetone;

(h) eluting the fraction eluted in step (g) on a silica gel column with a 20:10:1 to 5:10:1 gradient of toluene-acetone-formate mixture and collecting the fraction eluted with 10:10:1 toluene-acetone-formate;

(i) eluting the fraction eluted in step (h) on a silica gel column with a 30:1 to 1:1 gradient of dichloromethane/methanol mixture and collecting the fraction eluted with 20:1 dichloromethane/methanol; and

(j) recrystallizing the fraction eluted in step (i) with ethanol, thereby producing the coumestans compounds of formula I as precipitates

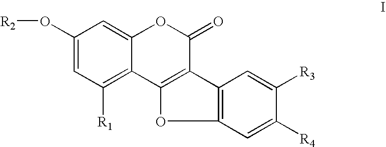

wherein,

R₁ represents H, OH, or methoxyl;

R₂ represents H, OH, or C₁-C₈ alkyl;

R₃ and R₄ are each independently selected from the group consisting of H, halogen, OH, and methoxyl.

In the third aspect, the invention provides a dietary supplement comprising 0.05-50wt % (preferably 0.1-10 wt %) of the compound of formula I, or the pharmaceutically acceptable salts thereof:

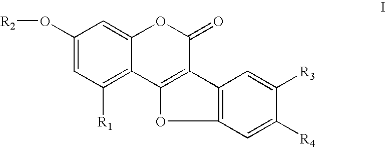

wherein

R₁ represents H, OH, or methoxyl;

R₂ represents H, OH, or C₁-C₈ alkyl;

R₃ and R₄ are each independently selected from the group consisting of H, halogen, OH, and methoxyl;

and a bromatologically acceptable carrier.

In the fourth aspect, the invention provides a pharmaceutical composition for treating arthritis, comprising:

(a) 0.05-90wt % compound of formula I or pharmaceutically acceptable salts thereof, or extracts containing the coumestans compound of formula I or pharmaceutically acceptable salts thereof, as the main active ingredient,

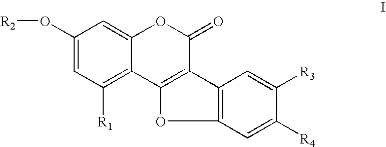

wherein

R₁ represents H, OH, or methoxyl; and

R₂ represents H, OH, or C₁-C₈ alkyl;

R₃ and R₄ are each independently selected from the group consisting of H, halogen, OH, and methoxyl; and

(b) one or more active ingredients selected from the group consisting of: acemetacin, diclofenac, ibuprofen, indomethacin, meloxicam, ketoprofen, sulindac, auranofin, naproxen, nabumetone, piroxicam, mecolfenamic acid, chlofenamic acid, mefenamic acid, pirprofen, fenbufen, tolmetin, flufenamide acid, methocarbamol, nimesulide, celecoxib, rofecoxib, aceclofenac methotrexate, gold salts, salazosulfadimidine, penicillamine, chloroquine, tripterygium wilfordii, ciclosporin, cyclophosphamide, and glococorticoids; and

(c) a pharmaceutically acceptable carrier.

### DESCRIPTION OF FIGURES

FIG. 1 shows a rat with arthritis caused by an adjuvant. The primary inflammation and edema of the left foot and the secondary inflammation and edema of the right foot are observed.

FIG. 2 shows the arthritis caused by an adjuvant, which is treated with MTX. Both the primary inflammation of the left foot and the secondary inflammation of the right foot are alleviated.

FIG. 3 shows the arthritis caused by an adjuvant, which is treated with wedenolactone (12.5 mg/kg). Both the primary inflammation of the left foot and the secondary inflammation of the right foot are alleviated.

FIG. 4 shows the arthritis caused by an adjuvant, which is treated with wedenolactone (0.25 mg/kg). Both the primary inflammation of the left foot and the secondary inflammation of the right foot are prone to be alleviated.

### MODE OF CARRYING OUT THE INVENTION

Through extensive and intensive studies, the inventor discovered that coumestans compounds of formula I or the pharmaceutically acceptable salts thereof, or the plant extract containing the coumestans compounds of formula I or the pharmaceutically acceptable salts can effectively treat arthritis,

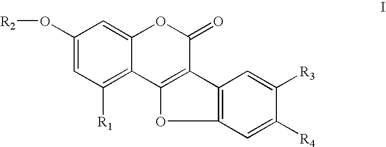

wherein

R₁ represents H, OH, or methoxyl;

R₂ represents H, OH, or C₁-C₈ alkyl;

R₃ and R₄ are each independently selected from the group consisting of H, halogen, OH, and methoxyl. The present invention is based on these discoveries.

The compound of formula I can be obtained by total- or semi-chemical synthesis, or by biological conversion. For example, it can be extracted from Eclipta prostrata Linn, Wedelia chinensis, or Eclipta alba. The compound can be extracted from the branches, leaves, or fruits of Compositae, but particularly from the leaves.

In 1956, Govindachari et al. for the first time isolated coumestans compound wedelolactone from Wedelia calendulacea, having the formula: 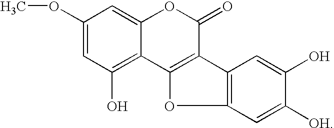
(Govindachari et al. “Chemical Examination of Wedelia Calendulacea, Part I, Structure of Wedelolactone”, Journal of the Chemical Society (1956), pp. 629-632.; Govindachari et al. “Chemical Investigation of Wedelia Calendulacea, Part II, The Position of the Methoxyl Group in Wedelolactone”, Journal of the Chemical Society, (1957),545-547; ).

Later, Bhargava et al. isolated desmethylwedelolactone from Eclipta alba, having the formula:

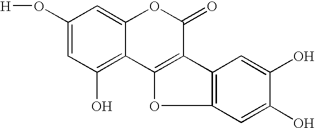

(Bhargava K K. et. al Isolation of desmethylwedelolactone and its glucoside from Eclipta alba. Indian J Chem, 1970,8(7):664-665 ).

Li C C. et al. and U.S. Pat. No.6,552,071 disclosed the structure and the chemical synthesis method of the derivatives of these compounds. (Li C C. et. al Total synthesis of wedelolactone. J Org Chem. Oct 31, 2003;68 (22):8500-4.) (Yuan et al. Methods for treating cell death diseases and inflammation. U.S. Pat. No. 6,552,071).

This class of compounds was considered to be useful for protecting liver, hemostasis, and anti-venoms. (Wong et al. “Wedelolactone and coumestan derivatives as new antihepatotic and antiphlogistic principles. Arzneimittelforschung”. May 1998 38(5):661-5; Melo. et al. “Inhibition of the myotoxic and hemorrhagic activities of crotalid venoms by Eclipta prostrate extracts and constituents”. Toxicon. May 1994;32(5):595-603).

As used herein, the compound of the invention refers to the compound of formula I:

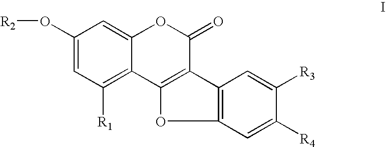

wherein

R₁ represents H, OH, or methoxyl;

R₂ represents H, OH, or C₁-C₈ alkyl;

R₃ and R₄ are each independently selected from the group consisting of H, halogen, OH, and methoxyl.

As used herein, “alkyl” refers to a straight or branched alkyl group having 1-8 carbon atoms, such as a methyl, ethyl, propyl, isopropyl, butyl, isobutyl, tertiary butyl, sec-butyl, amyl, neopentyl, hexyl, heptyl, or octyl group, preferably, straight or branched alkyl group having 1-4 carbon atoms, more preferably, methyl group.

The compound of this invention can be used in a form of pharmaceutically or physiologically acceptable salts formed with acid or base. The acid includes, but not limited to, chlorhydric acid, hydrobromic acid, sulfuric acid, citric acid, tartaric acid, phosphoric acid, lactic acid, pyruvic acid, acetic acid, succinic acid, oxalic acid, fumaric acid, maleic acid, ketosuccinic acid, methane-sulfonic acid, ethyl-sulfonic acid, benzene sulfonic acid, isethionic acid. The halide may also be used. Other salts include those derived from alkali metal or alkali earth metal (such as sodium, potassium, calcium, or magnesium), as well as those in the forms of ester, carbamate, or other conventional “prodrugs” (which will be converted into active forms in body).

An extract containing the compound of formula I can also be used in this invention. A preferred method of extraction is described above. Normally, the purity of the compound of formula I in the extract is 40-99.9 wt %, preferably, 50-98wt %.

The present invention also includes pharmaceutical compositions and methods for treating arthritis, which include administering a pharmaceutically effective amount of the compound of the invention to mammals.

The coumestans in this invention can be used for treating arthritis. Exemplary examples of arthritis include, but not limited to, rheumatic or rheumatoid arthritis, or osteoarthritis.

Pharmaceutical Compositions

The pharmaceutical compositions of this invention include a safe and effective amount of the compounds of this invention and a pharmaceutically acceptable carrier. “Safe and effective amount” means an amount that is effective to improve the condition being treated but not to cause serious side effects, defined in commonly acceptable medical terms. The safe and effective amount of a compound shall be determined by the factors such as the specific condition being treated, the age and physical condition of the patient, the severity of the disease, the duration of the treatment, the pharmaceutical carrier, and the administration route. The pharmaceutical composition contains about 0.1 -99.9wt % of the compound.

Typically, when the compound of the invention is used in the applications described above, the compound can be combined with one or more pharmaceutically acceptable carriers or excipients, such as solvents and diluents. They can be administered orally in the forms of tablet, capsule, dispersible powder, granule, suspension (e.g. containing about 0.05-5% suspension medium), syrup (e.g. containing about 10-50% sugar), elixir (e.g. containing about 20-50% ethanol), or administered parenterally in the forms of sterile injectable solution or suspension (containing about 0.05-5% suspension medium in isotonic medium). For example, these pharmaceutical preparations may contain about 2.5-90 wt %, typically 5%-60wt % active ingredient mixed with the carriers.

Common formulations include granule, powder, tablet, capsule, syrup, suppository, injection, emulsion, tincture, suspension, or solution, which are administered orally or non-orally.

For oral administration, the formulation may be in the forms of tablet, lozenge, capsule, pill, powder, granule, paste, suspension, emulsion, or solution.

For parenteral administration, the formulation may be in the forms of injection or infusion.

For intrajoint injection, the formulation may be in the form of suitable mixed suspension.

For intramuscular injection, the formulation may be in the form of aqueous solution, oil solution, mixed suspension or suitable storage formulation.

For local administration, the formulation may be in the form of lotion, cream, or gelata.

The effective amount of the active ingredients may vary depending on the administration pattern and severity of the disease being treated. Typically, however, desirable result can be achieved when the compound of the invention is administered in 0.5-500 mg/kg body, preferably in 2-4 separate doses daily or in a slow-releasing form. For most of the large mammals, the total daily dose is about 1-100 mg. The formulation suitable for oral administration is 0.5-500 mg active ingredients mixed with solid or liquid pharmaceutically acceptable carriers. The dosage can be adjusted to optimize the therapeutic response. For example, if required by the condition being treated, multiple separated doses may be administered daily, or the dose may be proportionally reduced. In general, the daily oral dose for adults is 1-1000 mg, preferably 10-200 mg; the daily non-oral dose for adults is 0.1-100 mg, preferably 1-100 mg.

In another preferred embodiment, the compounds of the invention may be administered orally, intravenously, intramuscularly, or subcutaneously. The solid carriers include amyloid, lactose, calcium monohydrogen phosphate, microcrystalline cellulose, saccharose, and kaolin; the liquid carriers include sterile water, polyethylene glycol, nonionic surfactant, and edible oil (corn oil, peanut oil, and gingili), as long as they are compatible with the active ingredients and the desired specific administration method. In the preparation, the adjuvants commonly used the pharmaceutical compositions, such as flavoring agents, pigments, preservatives, and antioxidants such as vitamin E and C, BHT and BHA, may also be advantageously included.

As used herein, “non-orally” means subcutaneous injection, intravenous injection, intraperitoneal injection, or drip transfusion. The injection preparations, such as sterile water or oil-based suspension preparations, can be obtained according to the conventional methods in the art using suitable dispersing agents, lubricants, or suspending agents. Sterile injection preparations are the non-toxic and non-oral solutions (such as water solutions) or suspensions in diluents or solutions. The carriers or solvents include water, isotonic saline, non-toxic and non-volatile oil. Any non-volatile oil or fatty acids, including natural, synthetic, or semi-synthetic fatty oil, fatty acids, and mono-, di- and tri-glycerides may be used.

The suppositories for rectal administration can be prepared by mixing the drug with a suitable non-irritating excipient such as cacao butter or PEG, which is in a solid state at room temperature, but become liquid in the intestine to release the drug into the rectum.

The oral solid drug preparations that is described above includes powder, granule, tablet, pill, and capsule. These dosage forms can be prepared by mixing the active ingredients with at least one of the following additives: sucrose, lactose, cellulose, manicol, maltose, dextran, starch, agar, alginate, chitin, chitosan, pectin, gum tragacanth, arabic gum, glutin, collagen, casein, albumin, and synthetic or semi-synthetic polymers and glycerides. These dosage forms can also contain additional additives, including inert diluents, lubricants such as magnesium stearate; preservatives such as p-hydroxyl benzoate and sorbic acid; anti-oxidants such as vitamin C, α-vitamin E and cysteine, disintegrants, binders, thickening agents, buffer, sweetening agents, flavouring agents, and aromatizers. Tablets and pills can also be coated. Oral liquid dosages include medicinal emulsion, syrup, tincture, suspension, and solution. They can contain commonly used inert diluents such as water.

From the point of simple drug manufacture and drug delivery, the preferred pharmaceutical compositions are solid compositions, particularly tablets and capsules filled with solid or liquid. Oral administration of the compound is preferred.

When the compound of the present invention is used to treat rheumatic or rheumatoid arthritis or osteoarthritis, it can be used in combination with one or more agents or treatments used for treating rheumatic or rheumatoid arthritis or osteoarthritis. For example, the compound may be used in combination with one or more of the active ingredients selected from the group consisting of:

NSAIDs: acemetacin, diclofenac, ibuprofen, indomethacin, meloxicam, ketoprofen, sulindac, auranofin, naproxen, nabumetone, piroxicam, mecolfenamic acid, chlofenamic acid, mefenamic acid, pirprofen, fenbufen, tolmetin, flufenamide acid, fenoprofen, methocarbamol, nimesulide, celecoxib, rofecoxib, and aceclofenac;

DMARDs: methotrexate, gold preparation, salazosulfadimidine, penicillamine, chloroquine, tripterygium wilfordii, ciclosporin, and cyclophosphamide, methotrexate, or

Corticosteroids: glucocorticoids such as cortisone and prednisolone.

The major advantages of the invention includes:

The invention discloses a class of coumetans compounds, which can effectively prevent and treat rheumatic and rheumatoid arthritis and osteoarthritis. In comparison with the existing therapeutic agents, the compounds of the present invention are more effective and less toxic.

The compounds in this invention can be used alone or in combination with other therapeutic agents as active ingredients in the medicaments or dietary supplements for preventing or treating arthritis.

The invention is further illustrated by the following examples. It is appreciated that these examples are only intended to illustrate the invention, but not to limit the scope of the invention. For the experimental methods in the following examples, they are performed under routine conditions or as instructed by the manufacturers, unless otherwise specified.

### EXAMPLE 1

Extraction of wedelolactone from Eclipta alba.

(1) Soak and Filtration

300 kg dried Eclipta alba. were soaked in 750 kg 95% ethanol overnight (10 hours), primarily filtered to remove the precipitates which were saved for further use, and secondarily filtered under vacuum or centrifugation (10000 rpm, 10 minutes) to remove dust and fine precipitates. Green and clear supernatant was recovered.

(2) Recovery of Ethanol

Ethanol was recovered by distillation at less than 60° C. The concentrated extracts were collected into a container in each 2 h reflux (the extract was dark-green in color and was ropy). This step was repeated until ethanol was completely recovered.

(3) Secondary Soak and Reflux

The recovered 750 kg ethanol was used to soak the precipitates obtained from step 1 overnight. Primary and secondary filtrations were performed followed by recovery through evaporation as described in step 2. The concentrated extract was collected.

(4) Extraction with Acetic Ester

50 volumes 50-80° C. (preferably 60-70° C.) hot water was added to the concentrated extracts from step 2 and 3. Vortex. The hot water phase was obtained through filtration by suction. Equal volume of acetic ester for extraction was added. It was mixed well and left at room temperature until the two phases were completely separated. The acetic ester phase was removed and collected, then distilled to dryness under vacuum at 50° C. and at reduced pressure. Small volume of ethanol was added to dissolve the dried extract, and then the extract was left at 4° C. overnight. The precipitate appeared and was collected by suction under reduced pressure, dried at 50° C. to produce the raw product.

(5) Separation and Purification of the Product

5 g above raw product was mixed with 10 g silica gel of 200-300 mesh and subjected to column (200 g, 200-300 mesh) chromatography, eluting with the gradient of petroleum ether-acetone solution. Each 100 mL fraction was collected together to obtain different polar fractions. The fractions containing the spot with Rf ⅓ (TLC in the mixture of 1:1 petroleum ether-acetone) were combined. The combined fractions were concentrated and again subjected to the silica gel column chromatography, eluting with a gradient of dichloromethane-acetone solution. Each 50 mL fraction was collected together to obtain different polar fractions. The fractions containing the spot with Rf ⅙ (TLC in the mixture of 3:1 dichloromethane-acetone) were combined. The combined fractions were concentrated and again subjected to the silica gel column chromatography, eluting with a gradient of methylbenzene-acetone-formate solution. Each 50 mL fraction was collected together to obtain different polar fractions. The fractions containing the spot with Rf ½ (TLC in the mixture of 10:10:1 methylbenzene-acetone-formate) were combined. The combined fractions were concentrated and subjected to the silica gel column chromatography, eluting with a gradient of dichloromethane-methanol solution. Each 25 mL fraction was collected together to obtain different polar fractions. The fractions containing the spot with Rf ⅙ (TLC in the mixture of 20:1 dichloromethane-methanol) were combined to produce the product with a purity of >90%. The yield was 1%.

(6) Preparation of the Standard Substance

30 mg of the above product with >90% purity was dissolved in 70% methanol, purified by reverse-phase column Lichroprep RP-18 (40-63μ), analyzed with TLC. The eluted fraction was collected and combined to produce the standard substance with a purity of >98%. The yield was about 90%. The appearance, molecular formula, melting point of the standard substance, and its IR, EIMS, ¹HNMR, ¹³CNMR peak values are as follows:

gray-white powder, molecular formula: C₁₆H₁₀O₇, mp 315° C. (dec.), UV λmax(MeOH, nm): 211.5(4.65), 247(4.40), 304(4.01)(sh), 350(4.48). IR(KBr) cm¹ 3300, 1715, 1640, 1620, 1445, 1415, 1320, 1205, 1155, 1070. EIMS m/z (%): 314(M⁺, 100), 313(22), 299([M-CH₃], 28), 285(5), 271([M-CH₃—CO], 8), 243([M-CH₃—CO—CO], 28), 187(17), 69(42). ¹HNMR(δ): 7.23 (s), 714 (s), 6.58 (d, J=2.3 Hz), 6.42 (d, J=2.3 Hz), 3.90(s)

¹³CNMR(δ): 158.0(C-1), 101.1(C-2), 159.6(C-3), 95.6(C-4), 99.3(C-5), 161.1(C-6), 95.0(C-7), 155.5(C-8), 155.0(C-9), 104.7(C-10), 145.2(C-11), 144.3(C-12), 99.0(C-13), 114.0(C-14), 148.7(C-15), 55.7(C-16).

The above data indicates that the obtained compound is wedelolactone, having the formula II:

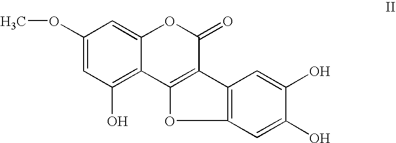

### EXAMPLE 2

### Inhibitory Effect of Compound Wedelolactone on the Mice Ear Inflammation Caused by Dimethylbenzene

Experimental animal: Kunming mice, body weight 18-22 g.

Method of administration: intraperitoneal injection. The doses of wedelolactone were 12.5 mg/kg and 25 mg/kg, respectively; the negative control used 0.5% CMC; the positive control used ibuprofen (25 mg/kg).

Method of experiment: The animals were divided randomly into 4 groups based on their body weights, 10 mice each group. The mice were administered with the test compound for 4 continuous days. On Day 4, both sides of the right ears of the mice were daubed evenly with 50 μl dimethylbenzene 2 hours after the administration. The left ears were not treated. The mice were left for 2 hours and then sacrificed by cervical dislocation. The ear specimens were obtained by punching both ears with a 8 mm hole-puncher. The specimens were weighed and the differences between the left and the right ears were used to evaluate the effect of the compound on the inflammation.

Results:

Table 1 demonstrates the result of the experiment, which shows that wedelolactone could inhibit the inflammation of the mice ear caused by dimethylbenzene.

TABLE 1
The effects of wedelolactone on the acute inflammation of mice
ear caused by dimethylbenzene. (mg, x̅ ± SD)

| | | | Difference |
|---|---|---|---|
| | | Animal | (mg) between |
| | Dose | number | left and |
| Group | (mg · kg⁻¹) | (n) | right ear |
| 0.5% CMC | — | 10 | 18.3 ± 3.3 |
| ibuprofen | 25 | 10 | 11.4 ± 3.6** |
| wedelolactone-12.5 mg/kg | 12.5 | 10 | 11.3 ± 3.6** |
| wedelolactone-25 mg/kg | 25 | 10 | 10.9 ± 4.6** |

**p < 0.01

### EXAMPLE 3

### The inhibitory Effects of Compound of Formula I on Rat Toes Inflammation Caused by Carrageenan

The compounds 1, 2, and 3 were synthesized according to Wong et al. (Wong et al “Wedelolactone and coumestan derivatives as new antihepatotic and antiphlogistic principles”. Arzneimittelforschung. May 1998 38(5):661-5;).

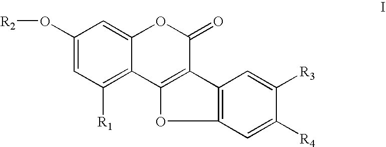

wherein, in compound 1: R₁═R₂═H R₃═R₄═OH

In compound 2: R₁═OH R₂═CH₃ R₃═R₄═OH

In compound 3: R₁═OH R₂═CH₃ R₃═Cl R₄═OH

Experimental animal: Male Wistar rats. Body weights are about 200 g.

Method of administration: intraperitoneal injection. The dose of compound 1, 2, or 3 is 12 mg/kg, respectively. The negative control used 0.5% CMC; the positive control used ibuprofen, with a dose of 25 mg/kg.

Method of experiment: The animals were divided randomly into 4 groups based on their body weights, 10 mice per group. The 4 groups were negative control group, positive control group, compound 1 group, compound 2 group, and compound 3 group. The animals were administered daily for 7 continuous days. On Day 7, the aponeurosis of right hind toes were injected with 0.1 ml of 1% carrageenan 30 minutes after the administration. The edema of the toes at different time points after induction of the inflammation was then measured.

Results:

The data in table 2 demonstrated that compounds 1, 2, and 3 could inhibit the edema of rat toes caused by carrageenan.

TABLE 2
The effects of the compounds of formula (I) on rat toes edema
caused by carrageenan (n = 10, x̅ ± SD).

| | Edema index (ml) after induction of the inflammation | | | | |
|---|---|---|---|---|---|
| Group | 1 h | 2 h | 4 h | 6 h | 24 h |
| 0.5% CMC | 0.46 ± 0.07 | 0.51 ± 0.11 | 0.59 ± 0.10 | 0.61 ± 0.07 | 0.51 ± 0.09 |
| ibuprofen | 0.31 ± 0.06*** | 0.38 ± 0.10*** | 0.42 ± 0.11*** | 0.39 ± 0.06*** | 0.37 ± 0.07*** |
| Compound 1 | 0.38 ± 0.05*** | 0.43 ± 0.06** | 0.47 ± 0.09*** | 0.53 ± 0.08** | 0.43 ± 0.07** |
| Compound 2 | 0.29 ± 0.06*** | 0.37 ± 0.09*** | 0.42 ± 0.07*** | 0.40 ± 0.05*** | 0.39 ± 0.08*** |
| Compound 3 | 0.31 ± 0.06*** | 0.34 ± 0.09*** | 0.38 ± 0.07*** | 0.42 ± 0.05*** | 0.41 ± 0.08*** |

***p < 0.001, extremely significant difference relative to the negative control group(0.5% CMC).
**p < 0.01, very significant difference relative to the negative control group(0.5% CMC).

### EXAMPLE 4

### The Effects of Wedelolactone on Adjuvant-Induced Arthritis of Rats

Experimental animal: Male Wistar rats. Body weights were about 150 g.

Method of administration: intraperitoneal injection. The dose of wedelolactone was 0.25 mg/kg or 12.5 mg/kg, prepared in 0.5% CMC. The dose of methotrexate (MTX) is 0.2 mg/kg.

Method of experiment: The animals were divided randomly into 4 groups based on their body weights. They were adapted for 3 days before experiments. The left hind planta of the rats were injected subcutaneously with 0.1 ml CFA emulsion to induce inflammation. On the Day 10, after the administration of the adjuvant, the rats were injected intraperitoneally with 5 ml/kg test compound wedelolactone for 19 continuous days. On and after the day of administration of the adjuvant, the change of thickness of the four rat paws was measured daily using a vernier caliper, and the volume of the back paw were measured using a Plethysmometer. The data from day 5, 10, 15, and 19 after administration were used for statistic analyses.

Results:

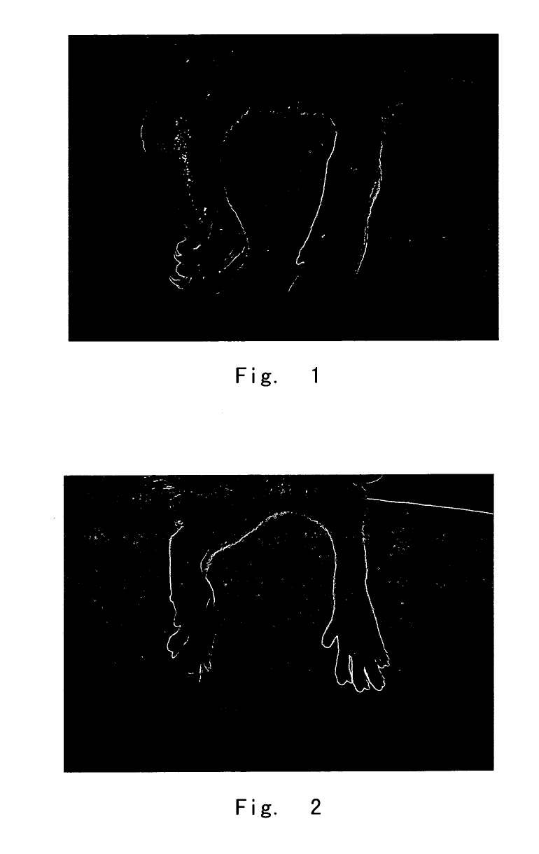

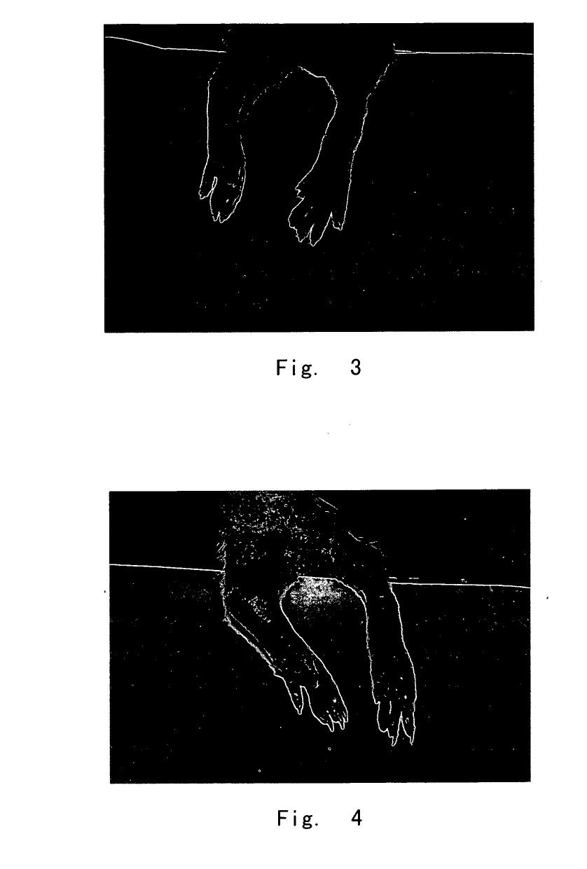

Data in table 3-8 and in FIG. 1-4 shows typical arthritis of rats and the comparison between the arthritis before and after treatment. The data demonstrated that wedelolactone could effectively inhibit rat adjuvant arthritis.

(1) Changes in the Paw Thickness of the Wistar Rats

TABLE 3
Left front paw thickness of Wistar rats on day 5, 10, 15,
and 19 after administration (mm, x̅ ± SD)

| group/thickness(left | Animal | | | | |
|---|---|---|---|---|---|
| front paw) | number | Day 5 | Day 10 | Day 15 | Day 19 |
| 0.5% CMC | 10 | 4.24 ± 0.19 | 4.71 ± 0.25 | 4.86 ± 0.37 | 4.90 ± 0.24 |
| MTX | 10 | 4.14 ± 0.14 | 3.96 ± 0.10** | 4.04 ± 0.10** | 4.09 ± 0.11** |
| wedelolactone-0.25 | 10 | 4.20 ± 0.11 | 4.71 ± 0.24 | 4.61 ± 0.10 | 4.64 ± 0.17** |
| wedelolactone-12.5 | 10 | 4.11 ± 0.11 | 4.03 ± 0.13** | 4.11 ± 0.16** | 4.04 ± 0.14** |

**p < 0.01, very significant difference relative to the negative control group(0.5% CMC).

TABLE 4
Right front paw thickness of Wistar rats on day 5, 10, 15,
and 19 after administration (mm, x̅ ± SD)

| group/thickness(right | Animal | | | | |
|---|---|---|---|---|---|
| front paw) | number | Day 5 | Day 10 | Day 15 | Day 19 |
| 0.5% CMC | 10 | 4.17 ± 0.22 | 4.66 ± 0.36 | 4.79 ± 0.39 | 4.89 ± 0.31 |
| MTX | 10 | 4.00 ± 0.10 | 3.84 ± 0.10** | 4.07 ± 0.13** | 4.00 ± 0.12** |
| wedelolactone-0.25 | 10 | 4.13 ± 0.15 | 4.66 ± 0.09 | 4.54 ± 0.15 | 4.59 ± 0.15* |
| wedelolactone-12.5 | 10 | 4.01 ± 0.17 | 3.95 ± 0.12** | 4.11 ± 0.16** | 4.04 ± 0.07** |

**p < 0.01, very significant difference relative to the negative control group(0.5% CMC).
*p < 0.05, significant difference relative to the negative control group(0.5% CMC).

TABLE 5
Left hind paw thickness of Wistar rats on day 5, 10, 15, and
19 after administration (mm, x̅ ± SD)

| group/thickness | Animal | | | | |
|---|---|---|---|---|---|
| (left hind paw) | number | Day 5 | Day 10 | Day 15 | Day 19 |
| 0.5% CMC | 10 | 7.26 ± 0.85 | 8.64 ± 0.69 | 8.83 ± 0.53 | 9.07 ± 0.28 |
| MTX | 10 | 6.96 ± 1.97 | 7.44 ± 1.42 | 7.86 ± 1.36 | 7.51 ± 1.40* |
| wedelolactone-0.25 | 10 | 7.10 ± 0.83 | 8.18 ± 1.22 | 8.51 ± 0.91 | 8.49 ± 1.06 |
| wedelolactone-12.5 | 10 | 7.13 ± 0.96 | 8.09 ± 1.05 | 8.44 ± 1.63 | 8.53 ± 1.33 |

*p < 0.05, significant difference relative to the negative control group(0.5% CMC).

TABLE 6
Right hind paw thickness of Wistar rats on day 5, 10, 15,
and 19 after administration (mm, x̅ ± SD)

| group/thickness | Animal | | | | |
|---|---|---|---|---|---|
| (right hind paw) | number | Day 5 | Day 10 | Day 15 | Day 19 |
| 0.5% CMC | 10 | 5.2 ± 0.23 | 5.44 ± 0.30 | 5.46 ± 0.54 | 5.57 ± 0.20 |
| MTX | 10 | 4.89 ± 0.23* | 4.87 ± 0.18** | 5.04 ± 0.28 | 4.90 ± 0.08** |
| wedelolactone-0.25 | 10 | 5.05 ± 0.24 | 5.28 ± 0.38 | 5.15 ± 0.35 | 5.23 ± 0.23** |
| wedelolactone-12.5 | 10 | 4.95 ± 0.27 | 5.05 ± 0.25* | 5.20 ± 0.23 | 4.98 ± 0.10** |

**p < 0.01, very significant difference relative to the negative control group(0.5% CMC).
*p < 0.05, significant difference relative to the negative control group(0.5% CMC).

(2) Changes in Volume of the Hind Paws of Wistar Rat

TABLE 7
Changes in left hind paw volume of Wistar rats on
day 5, 10, 15, and 19 after administration (ml)

| group/volume | Animal | | | | |
|---|---|---|---|---|---|
| (left hind paw) | number | Day 5 | Day 10 | Day 15 | Day 19 |
| 0.5% CMC | 10 | 1.90 ± 0.24 | 2.55 ± 0.25 | 2.89 ± 0.43 | 2.70 ± 0.44 |
| MTX | 10 | 1.68 ± 0.32 | 1.73 ± 0.23** | 1.93 ± 0.34** | 1.84 ± 0.26** |
| wedelolactone-0.25 | 10 | 1.85 ± 0.24 | 2.38 ± 0.30 | 2.63 ± 0.37 | 2.61 ± 0.42 |
| wedelolactone-12.5 | 10 | 1.64 ± 0.31 | 2.14 ± 0.45* | 2.42 ± 0.62 | 2.46 ± 0.75 |

**p < 0.01, very significant difference relative to the negative control group(0.5% CMC).
*p < 0.05, significant difference relative to the negative control group(0.5% CMC).

TABLE 8
Changes in right hind paw volume of Wistar rats on
day 5, 10, 15, and 19 after administration (ml)

| group/volume | Animal | | | | |
|---|---|---|---|---|---|
| (right hind paw) | number | Day 5 | Day 10 | Day 15 | Day 19 |
| 0.5% CMC | 10 | 1.19 ± 0.08 | 1.29 ± 0.09 | 1.47 ± 0.24 | 1.55 ± 0.17 |
| MTX | 10 | 1.09 ± 0.07* | 1.00 ± 0.10** | 1.17 ± 0.11* | 1.19 ± 0.09** |
| wedelolactone-0.25 | 10 | 1.18 ± 0.05 | 1.16 ± 0.08* | 1.29 ± 0.13 | 1.36 ± 0.12* |
| wedelolactone-12.5 | 10 | 1.10 ± 0.10 | 1.03 ± 0.10** | 1.24 ± 0.16 | 1.19 ± 0.08** |

**p < 0.01, very significant difference relative to the negative control group(0.5% CMC).
*p < 0.05, significant difference relative to the negative control group(0.5% CMC).

### EXAMPLE 5

### Effects of Wedelolactone on Collagen-Induced Rat Arthritis

Experimental animal: Male Wistar rats. Body weights were about 120 g.

Administration method: intraperitoneal injection. The dose of wedelolactone is 6 or 12 mg/kg. Negative control used 0.5% CMC.

Experimental methods: 1). Preparation of type-II collagen. The fresh calf cartilages were ground at 4° C., immersed and washed with Tris-HCL₂. The left residue was digested with pepsin. The supernatant was collected and salted out, followed by chromatography with DE-52 column to obtain type-II collagen. The collagen were dissolved in glacial acetic acid and mixed with Freund's adjuvant by grounding. Each of the animals, except the controls, was subcutaneously injected with a total of 2.5 mg/kg collagen at multiple sites of the tail root and back neck. 7 days thereafter, the animals were boostered by subcutaneous administration with 1.0 mg/kg collagen in the tail root and back neck. The immunized animals were divided randomly into 4 groups based on their body weight, 10 mice in each group. The four groups were modeling blank group, modeling control group, low dose wedelolactone group, and high dose wedelolactone group. The daily administration started at day 10 after the first immunization for 3 weeks continuously. The effects of the drug on the rat rheumatoid arthritis and the delayed-hypersensitivity were recorded. Starting from day 10 after the first immunization, the following data were recorded every 10 days: A) arthritis surface temperature (the surface temperature of both hind leg joints were measured by using a digital temperature recorder, and the temperature differences before and after the immunization was calculated). B) arthritis score (arthritis was scored following the method described in the literature. 1 point: inflammation in one area of paws or planta; 2 points: inflammation in more than two areas of paws or planta; 3 points: mild inflammation in the whole legs; 4 points: severe inflammation causing joint rigidity, deformation and dysfunction. The highest score for each paw is 4 points. The highest total score for each rat is 16 points. C) Delayed-hypersensitivity measurement. At day 24, 10 μl type II collagen containing solution was injected subcutaneously in the back of the right ear of each rat. The thickness of the ear before and 48 hr after the injection was measured.

Results:

The data in table 9-12 demonstrates that wedelolactone can effectively inhibit the collagen-induced rat arthritis.

TABLE 9
The effects of wedelolactone on the ankle joint surface temperature
of the collagen-induced arthritic rats ( x̅ ± SD)

| | | Temp. before | Joint surface temperature difference (° C.) at | | |
|---|---|---|---|---|---|
| | Joint | immunization | different time points | | |
| Group | number | (° C.) | Day 10 | Day 20 | Day 30 |
| modeling blank | 20 | 27.40 ± 0.75 | −0.01 ± 0.36*** | 0.01 ± 0.37*** | 0.01 ± 0.37*** |
| modeling control | 20 | 27.32 ± 0.68 | 1.35 ± 0.65 | 1.60 ± 0.62 | 1.76 ± 0.81 |
| wedelolactone-6 | 20 | 27.29 ± 0.72 | 1.42 ± 0.74* | 0.83 ± 0.44*** | 0.72 ± 0.40** |
| wedelolactone-12 | 20 | 27.36 ± 0.66 | 1.36 ± 0.50* | 0.73 ± 0.45*** | 0.48 ± 0.39*** |

***p < 0.001, extremely significant difference relative to the negative control group(0.5% CMC).
**p < 0.01, very significant difference relative to the negative control group(0.5% CMC).
*p < 0.05, significant difference relative to the negative control group(0.5% CMC).

TABLE 10
The effects of wedelolactone on inflammation score of the collagen
II-induced arthritic rats ( x̅ ± SD)

| | | inflammation score at | | |
|---|---|---|---|---|
| | Animal | different time points | | |
| Group | number | Day 10 | Day 20 | Day 30 |
| modeling | 10 | 0.00 ± 0.00*** | 0.00 ± 0.00*** | 0.00 ± 0.00*** |
| blank | | | | |
| modeling | 10 | 3.80 ± 1.90 | 5.60 ± 1.85 | 5.90 ± 1.92 |
| control | | | | |
| wedelo- | 10 | 4.00 ± 2.02* | 3.10 ± 1.45*** | 2.7 ± 1.35*** |
| lactone-6 | | | | |
| wedelo- | 10 | 4.10 ± 2.08* | 2.00 ± 1.41*** | 1.40 ± 1.02*** |
| lactone-12 | | | | |

***p < 0.001, extremely significant difference relative to the negative control group(0.5% CMC).
**p < 0.01, very significant difference relative to the negative control group(0.5% CMC).
*p < 0.05, significant difference relative to the negative control group(0.5% CMC).

TABLE 11
The effects of wedelolactone on the delayed hypersensitivity of the
collagen II-induced arthritic rats ( x̅ ± SD)

| | | Thickness of | Increased |
|---|---|---|---|
| | | the right ear | thickness |
| | Animal | before | of the |
| Group | number | injection (mm) | right ear (mm) |
| modeling blank | 10 | 0.752 ± 0.101* | 0.002 ± 0.026*** |
| modeling control | 10 | 0.784 ± 0.072 | 0.403 ± 0.183 |
| wedelolactone-6 | 10 | 0.793 ± 0.071* | 0.231 ± 0.113** |
| wedelolactone-12 | 10 | 0.798 ± 0.070* | 0.194 ± 0.115*** |

***p < 0.001, extremely significant difference relative to the negative control group(0.5% CMC).
**p < 0.01, very significant difference relative to the negative control group(0.5% CMC).
*p < 0.05, significant difference relative to the negative control group(0.5% CMC).

### EXAMPLE 6

### Tablet Preparation

The following components were mixed by using conventional methods, and then compressed into tablets to produce the pharmaceutical composition in the form of tablet:

| Components | Quantity | |
|---|---|---|
| Wedelolactone of Example I | 20 | mg |
| Lactose | 130 | mg |
| Corn starch | 40 | mg |
| magnesium stearate | 10 | mg |
| Total | 200 | mg |

### EXAMPLE 7

### Tablet Preparation

The following components were mixed by using conventional methods, and then compressed into tablets to produce the pharmaceutical composition in the form of tablet:

| Components | Quantity | |
|---|---|---|
| Wedelolactone of Example I | 15 | mg |
| Ibuprofen | 5 | mg |
| Lactose | 130 | mg |
| Corn starch | 40 | mg |
| magnesium stearate | 10 | mg |
| Total | 200 | mg |

### EXAMPLE 8

### Tablet Preparation

The following components were mixed by using conventional methods, and then compressed into tablets to produce the pharmaceutical composition in the form of tablet:

| Components | Quantity | |
|---|---|---|
| Wedelolactone of Example I | 15 | mg |
| Hydrocortisone | 5 | mg |
| Lactose | 130 | mg |
| Corn starch | 40 | mg |
| magnesium stearate | 10 | mg |
| Total | 200 | mg |

All publications and patent applications mentioned in this specification are herein incorporated by reference to the same extent as if each independent publication or patent application was specifically and individually indicated to be incorporated by reference. In addition, it is understood that those skilled in the art can make various changes or modifications after reading the above teachings of the present invention. These equivalents are also falling into the scope defined by the claims.

What is claimed:

1. A use of the coumestans compound of formula I or pharmaceutically acceptable salts thereof, or an extract containing the coumestans compound of formula I or pharmaceutically acceptable salts thereof in the manufacture of a medicament for the treatment or prevention of arthritis 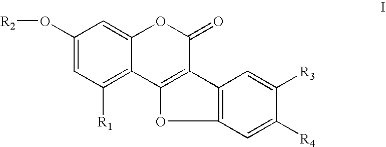
wherein
R₁ represents H, OH, or methoxyl;
R₂ represents H, OH, or C₁-C₈ alkyl;
R₃ and R₄ are each independently selected from the group consisting of H, halogen, OH, and methoxyl.

2. The use of claim 1, wherein the pharmaceutically acceptable salt of the compound of formula I are formed with the acids selected from the group consisting of chlorhydric acid, hydrobromic acid, sulfuric acid, citric acid, tartaric acid, phosphoric acid, lactic acid, pyruvic acid, acetic acid, succinic acid, oxalic acid, fumaric acid, maleic acid, ketosuccinic acid, methane-sulfonic acid, ethyl-sulfonic acid, benzene sulfonic acid, and isethionic acid.

3. The use of claim 1, wherein the compound of formula I or the extract is extracted from the plant Compositae.

4. The use of claim 3, wherein the plant Compositae is selected from Eclipta prostrata Linn, Wedelia chinensis, and Eclipta alba.

5. The use of claim 1, wherein said compound of formula I is wedelolactone as shown in formula II:

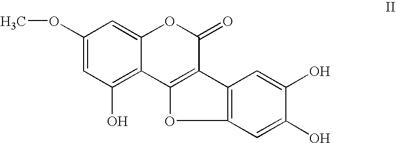

6. The use of claim 1, wherein the arthritis is selected from the group consisting of rheumatic arthritis, rheumatoid arthritis, and osteoarthritis.

7. The use of claim 1, wherein the compound of formula I is produced by a extraction method comprising the steps of:
(a) extracting the fruits, leaves, or branches of the Compositae plant with 95±3% ethanol, thereby producing ethanol extract;
(b) dissolving the above ethanol extract in 5-300 volumes of H₂O at 50-80° C., filtering to remove the precipitates and collecting the H2O phase;
(c) extracting the H₂O phase in step (b) with acetic ester and collecting the acetic ester phase;
(d) concentrating and drying the acetic ester phase in step (c) to produce the precipitates;
(e) eluting the precipitates in (d) on a silica gel column with a 5:1 to 1:2 gradient of petroleum ether/acetone mixture and collecting the fraction eluted with 1:1 petroleum ether/acetone;
(f) concentrating the fraction eluted in step (e) to produce concentrated residue;
(g) eluting the concentrated residue in step (f) on a silica gel column with a 5:1 to 1:2 gradient of dichloromethane/acetone mixture and collecting the fraction eluted with 3:1 dichloromethane/acetone;
(h) eluting the fraction eluted in step (g) on a silica gel column with a 20:10:1 to 5:10:1 gradient of toluene-acetone-formate mixture and collecting the fraction eluted with 10:10:1 toluene-acetone-form ate;
(i) eluting the fraction eluted in step (h) on a silica gel column with a 30:1 to 1:1 gradient of dichloromethane/methanol mixture and collecting the fraction eluted with. 20:1 dichloromethane/methanol; and
recrystallizing the fraction eluted in step (i) with ethanol, thereby producing the coumestans compounds of formula I as precipitates.

8. A method for producing coumestans, comprising the steps of:
(a) extracting the fruits, leaves, or branches of the Compositae plant with 95±3% ethanol, thereby producing ethanol extract;
(b) dissolving the above ethanol extract in 5-300 volumes of H₂O at 50-80° C., filtering to remove the precipitates and collecting the H₂O phase;
(c) extracting the H₂O phase in step (b) with acetic ester and collecting the acetic ester phase;
(d) concentrating and drying the acetic ester phase in step (c) to produce the precipitates;
(e) eluting the precipitates in (d) on a silica gel column with a 5:1 to 1:2 gradient of petroleum ether/acetone mixture and collecting the fraction eluted with 1:1 petroleum ether/acetone;
(f) concentrating the fraction eluted in step (e) to produce concentrated residue;
(g) eluting the concentrated residue in step (f) on a silica gel column with a 5:1 to 1:2 gradient of dichloromethane/acetone mixture and collecting the fraction eluted with 3:1 dichloromethane/acetone;
(h) eluting the fraction eluted in step (g) on a silica gel column with a 20:10:1 to 5:10:1 gradient of toluene-acetone-formate mixture and collecting the fraction eluted with 10:10:1 toluene-acetone-formate;
(i) eluting the fraction eluted in step (h) on a silica gel column with a 30:1 to 1:1 gradient of dichloromethane/methanol mixture and collecting the fraction eluted with 20:1 dichloromethane/methanol; and
recrystallizing the fraction eluted in step (i) with ethanol, thereby producing the coumestans compounds of formula I as precipitates 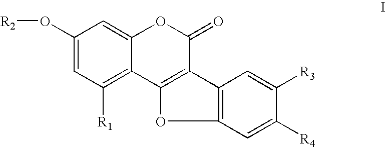
wherein,
R₁ represents H, OH, or methoxyl;
R₂ represents H, OH, or C₁-C₈ alkyl;
R₃ and R₄ are each independently selected from the group consisting of H, halogen, OH, and methoxyl.

9. A dietary supplement comprising 0.05-50wt % of the compound of formual I, or the pharmaceutically acceptable salts thereof: 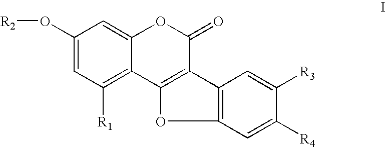
wherein
R₁ represents H, OH, or methoxyl;
R₂ represents H, OH, or C₁-C₈ alkyl;
R₃ and R₄ are each independently selected from the group consisting of H, halogen, OH, and methoxyl;
and a bromatologically acceptable carrier.

10. A pharmaceutical composition for treating arthritis, comprising:
(a) 0.05-90wt % compound of formula I or pharmaceutically acceptable salts thereof, or extracts containing the coumestans compound of formula I or pharmaceutically acceptable salts thereof, as the main active ingredient, 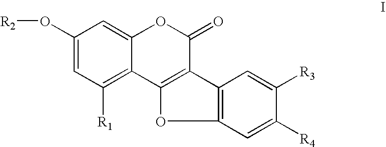
wherein
R₁ represents H, OH, or methoxyl; and
R₂ represents H, OH, or C₁-C₈ alkyl;
R₃ and R₄ are each independently selected from the group consisting of H, halogen, OH, and methoxyl; and
(b) one or more active ingredients selected from the group consisting of: acemetacin, diclofenac, ibuprofen, indomethacin, meloxicam, ketoprofen, sulindac, auranofin, naproxen, nabumetone, piroxicam, mecolfenamic acid, chlofenamic acid, mefenamic acid, pirprofen, fenbufen, tolmetin, flufenamide acid, methocarbamol, nimesulide, celecoxib, rofecoxib, aceclofenac methotrexate, gold salts, salazosulfadimidine, penicillamine, chloroquine, tripterygium wilfordii, ciclosporin, cyclophosphamide, and glococorticoids; and
(c) a pharmaceutically acceptable carrier.
